(19) United States
(12) Patent Application Publication
(10) Pub. No.: US 2024/0024109 A1
(43) Pub. Date: Jan. 25, 2024

(54) Enhancing Positional Adjustment of Devices that are Delivered via a Catheter

(71) Applicant: Edwards Lifesciences Corporation, Irvine, CA (US)

(72) Inventors: David ALON, Zichron Yaacov (IL); Dmitry PINHASOV, Yokneam Illit (IL)

(21) Appl. No.: 18/109,419

(22) Filed: Feb. 14, 2023

Related U.S. Application Data
(60) Provisional application No. 63/311,162, filed on Feb. 17, 2022.

(51) Int. Cl. A61F 2/24

(52) U.S. Cl. CPC A61F 2/2466; A61F 2/2445; A61M 25/0074

(57) ABSTRACT

An implant may be affixed to a cardiac valve annulus by an apparatus that includes a catheter with a first actuatable bending section and a support structure affixed to the distal end of the catheter. Support arms extend distally beyond the support structure. In some embodiments, a shaft with a second actuatable bending section is permanently positioned between the support arms, and an inflatable balloon surrounds at least a portion of the shaft. In other embodiments, a subassembly that includes a shaft with a second actuatable bending section and an inflatable balloon is slidably mounted so that it can move from an initial position that is spaced apart from the support structure to a second position where the balloon is between the at least four support arms. In either case, actuating the second bending section when the balloon is inflated will move the support arms that support the implant.

### CROSS REFERENCE TO RELATED APPLICATIONS

This application claims the benefit of U.S. Provisional Application 63/311,162, filed Feb. 17, 2022, which is incorporated herein by reference in its entirety.

### BACKGROUND

U.S. Pat. No. 10,786,356, which is incorporated herein by reference in its entirety, depicts a variety of approaches for delivering a constricting cord or a ring to a cardiac valve annulus and subsequently implanting the constricting cord or ring. In this context, precisely aligning the constricting cord or ring with the annulus prior to implantation can be very beneficial.

### SUMMARY OF THE INVENTION

One aspect of the invention is directed to a first apparatus for affixing a ring or cord to a cardiac annulus or adjacent tissue. The first apparatus comprises a steerable catheter, a support structure, at least four support arms, and a subassembly that is slidably disposed with respect to the support structure. The steerable catheter has a distal portion, a distal end, and a first actuatable bending section disposed in the distal portion of the catheter. The support structure is affixed to the distal end of the catheter, and the support structure includes at least one first mating feature. Each of the at least four support arms is affixed to the support structure and extends distally beyond the support structure, and each of the support arms is at least 2 cm long. The subassembly has a proximal segment and a proximal end, and the subassembly includes (a) a shaft that extends distally from the proximal segment, wherein the shaft has a second actuatable bending section, and (b) an inflatable balloon that surrounds at least a portion of the shaft. The proximal segment of the subassembly has at least one second mating feature that is shaped and dimensioned to form a keyed connection with the at least one first mating feature. The subassembly is positioned distally beyond the support structure and is slidably disposed with respect to the support structure. The support structure and the subassembly are disposed at respective initial positions at which the proximal end of the subassembly is spaced apart from the support structure by at least 1 cm.

In some embodiments of the first apparatus, the at least four support arms are configured to spring apart from each other upon sliding distally beyond a distal end of a sheath that is dimensioned to slidably surround the catheter, the at least four support arms, and the subassembly. The subassembly is configured for sliding in a proximal direction with respect to the catheter to a second position after the support arms have sprung apart from each other, such that when the subassembly has slid to the second position, (a) the at least one second mating feature makes the keyed connection with the at least one first mating feature and (b) at least a portion of the balloon is disposed between the at least four support arms. The balloon is configured so that when the subassembly is at the second position, inflation of the balloon causes the balloon to press outward against at least some of the support arms. Optionally, the embodiments described in this paragraph may further comprise the sheath.

In some embodiments of the first apparatus, the at least one first mating feature comprises at least one opening, and the at least one second mating feature comprises at least one member dimensioned to slidably fit into the at least one opening.

In some embodiments of the first apparatus, the at least one first mating feature comprises a plurality of openings, and the at least one second mating feature comprises a plurality of prongs dimensioned to slidably fit into the plurality of openings.

In some embodiments of the first apparatus, the at least one first mating feature comprises at least two lumens, and the at least one second mating feature comprises at least two flexible metal rods dimensioned to slide within the at least two lumens.

In some embodiments of the first apparatus, the first actuatable bending section has at least two degrees of freedom and the second actuatable bending section has at least one degree of freedom.

In some embodiments of the first apparatus, the first actuatable bending section has at least one actuator configured to bend the first actuatable bending section within a first plane and at least one actuator configured to bend the first actuatable bending section within a second plane that is offset by 60-120° from the first plane. The second actuatable bending section has at least one actuator configured to bend the second actuatable bending section within the first plane.

In some embodiments of the first apparatus, the first actuatable bending section has at least one pull wire configured to bend the first actuatable bending section within a first plane and at least one pull wire configured to bend the first actuatable bending section within a second plane that is offset by 60-120° from the first plane, and the second actuatable bending section has at least one pull wire configured to bend the second actuatable bending section within the first plane.

In some embodiments of the first apparatus, the support structure and the subassembly are disposed at respective initial positions at which the proximal end of the subassembly is spaced apart from the support structure by at least 3 cm. In some embodiments of the first apparatus, the shaft is at least 3 cm long. In some embodiments of the first apparatus, the catheter has an outer diameter less than 8 mm.

Some embodiments of the first apparatus further comprise the ring or cord, at least four anchors configured to affix the ring or cord to the cardiac annulus or adjacent tissue, and at least four anchor launchers. Each of the at least four anchors is configured to drive a respective one of the anchors into the cardiac annulus or adjacent tissue, and each of the at least four anchor launchers is supported by a respective one of the at least four support arms.

Another aspect of the invention is directed to a second apparatus for affixing a ring or cord to a cardiac annulus or adjacent tissue. The second apparatus comprises a steerable catheter, a support structure, at least four support arms, a shaft, and an inflatable balloon. The steerable catheter has a distal portion, a distal end, and a first actuatable bending section disposed in the distal portion of the catheter. The support structure is affixed to the distal end of the catheter. Each of the at least four support arms is affixed to the support structure and extends distally beyond the support structure, and each of the support arms is at least 2 cm long. The shaft extends distally from the support structure, and the shaft has a second actuatable bending section positioned between the at least four support arms. The inflatable balloon surrounds at least a portion of the shaft, and at least a portion of the balloon is positioned between the at least four support arms.

In some embodiments of the second apparatus, the at least four support arms are configured to spring apart from each other upon sliding distally beyond a distal end of a sheath that is dimensioned to slidably surround the catheter, the at least four support arms, the shaft, and the balloon. The balloon is configured so that inflation of the balloon causes the balloon to press outward against at least some of the support arms. Optionally, the embodiments described in this paragraph may further comprise the sheath.

In some embodiments of the second apparatus, the first actuatable bending section has at least two degrees of freedom and the second actuatable bending section has at least one degree of freedom.

In some embodiments of the second apparatus, the first actuatable bending section has at least one actuator configured to bend the first actuatable bending section within a first plane and at least one actuator configured to bend the first actuatable bending section within a second plane that is offset by 60-120° from the first plane. The second actuatable bending section has at least one actuator configured to bend the second actuatable bending section within the first plane.

In some embodiments of the second apparatus, the first actuatable bending section has at least one pull wire configured to bend the first actuatable bending section within a first plane and at least one pull wire configured to bend the first actuatable bending section within a second plane that is offset by 60-120° from the first plane. The second actuatable bending section has at least one pull wire configured to bend the second actuatable bending section within the first plane.

Some embodiments of the second apparatus further comprise the ring or cord, at least four anchors configured to affix the ring or cord to the cardiac annulus or adjacent tissue, and at least four anchor launchers. Each of the at least four anchors is configured to drive a respective one of the anchors into the cardiac annulus or adjacent tissue, and each of the at least four anchor launchers is supported by a respective one of the at least four support arms.

### BRIEF DESCRIPTION OF THE DRAWINGS

FIG. 1 depicts a first embodiment for delivering an implant (e.g., a constricting cord or ring) to a cardiac valve annulus.

FIG. 2 depicts a second embodiment for delivering an implant (e.g., a constricting cord or ring) to a cardiac valve annulus that provides improved maneuverability of the implant.

FIG. 3 depicts a third embodiment for delivering an implant (e.g., a constricting cord or ring) to a cardiac valve annulus that provides improved maneuverability of the implant at an initial point in time during which all of the components are squeezed together within a sheath.

FIG. 4 depicts the state of the third embodiment at a subsequent time when most of its components extend distally beyond the sheath.

FIG. 5 depicts the third embodiment at a subsequent time after a subassembly has slid in the proximal direction with respect to the catheter.

FIG. 6 is a cross-sectional view that corresponds to FIG. 5.

FIG. 7A is a detail of the interface between the subassembly and the support structure when the third embodiment is in the state depicted in FIG. 4.

FIG. 7B is a detail of the interface between the subassembly and the support structure when the third embodiment is in the state depicted in FIG. 5.

FIG. 7C depicts a detail of the female mating features depicted in FIGS. 7A-7B.

FIG. 7D depicts a detail of the male mating features depicted in FIGS. 7A-7B.

FIG. 8 depicts the state of the third embodiment after the balloon has been inflated so that it presses outward against the support arms.

FIG. 9 depicts the state of the third embodiment after the implant has been affixed to the annulus.

FIG. 10 depicts the third embodiment during withdrawal of the device into the access sheath.

Various embodiments are described in detail below with reference to the accompanying drawings, wherein like reference numerals represent like elements.

### DESCRIPTION OF THE PREFERRED EMBODIMENTS

Conventional steerable catheters can be very useful for aligning devices to the relevant target anatomy prior to implantation. But in some situations (e.g., due to the nature of the implantable device itself, the device's delivery mechanism, and/or the anatomic location of implantation), the device that is being implanted may be positioned a significant distance (e.g., >5 cm) away from the actuatable bending section of the catheter. In these situations (e.g., when installing a ring or a cinching cord onto a cardiac annulus), it can be difficult to precisely align the device to the target anatomy, even when the catheter's steering mechanism provides multiple degrees of freedom.

The embodiments described herein make it easier to precisely align the implantable device to the target anatomy by using a secondary actuatable bending section in conjunction with a primary actuatable bending section that is disposed within the catheter.

In some embodiments, a support structure is affixed to the distal end of a steerable catheter, and support arms (which hold the implant) extend distally beyond the support structure. A shaft extends distally from the support structure between the support arms, and the shaft has a secondary actuatable bending section that provides significant advantages, as described below.

In some embodiments, a support structure is affixed to the distal end of a steerable catheter, and support arms (which hold the implant) extend distally beyond the support structure. A secondary actuatable bending section is disposed in a subassembly that is (a) positioned distally beyond the support structure and (b) slidably disposed with respect to the support structure. Because the secondary bending section is disposed in a subassembly that is slidably disposed with respect to the support structure, and the support structure is affixed to the catheter, the spacing between the secondary bending section and the primary bending section can be adjusted. And this adjustability can provide additional significant advantages, as described below.

Note that as used herein, the terms “distal” and “proximal” are from the point of reference of the practitioner who is using the device (as opposed to the subject that is being treated). The distal end of the catheter is therefore the end that extends deepest into the subject's body, and the proximal end of the catheter will typically never enter the subject's body.

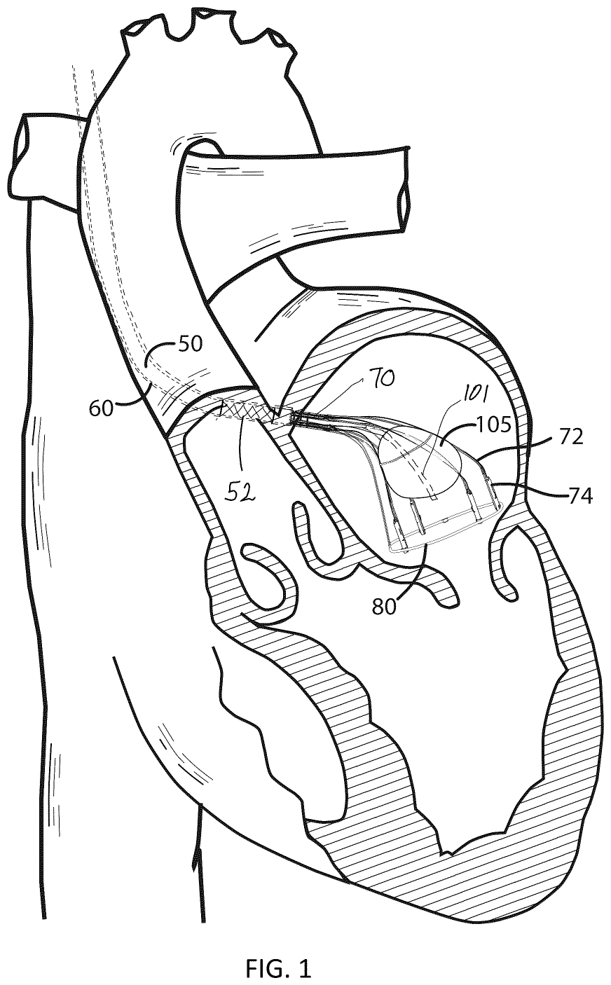

FIG. 1 depicts an apparatus for delivering a flexible implant 80 (e.g., a constricting cord or a flexible ring) to a cardiac valve annulus (e.g., the mitral valve annulus in the example illustrated in FIG. 1 or the tricuspid valve annulus). This apparatus includes a set of support arms 72 that extend distally beyond the end of a catheter 50, and the support arms 72 are affixed to the distal end of the catheter 50 by a support structure 70. One way for affixing the support arms 72 to the catheter 50 using an intervening support structure 70 is described in U.S. Pat. No. 10,786,356, and a variety of alternative approaches will be apparent to persons skilled in the relevant arts. A set of anchors are pre-affixed to the implant 80, and each of these anchors is driven into the annulus or adjacent tissues by a respective anchor launcher 74 that is supported by a respective one of the support arms 72.

FIG. 1 depicts the apparatus at a moment in time shortly before implantation of the implant 80 (i.e., before the anchor launchers 74 drive the anchors into the annulus). But prior to arriving at this state, the support arms 72, the anchor launchers 74, and the flexible implant 80 are all squeezed together within an access sheath 60, so that they can be delivered via a minimally invasive approach. After the apparatus is routed to the appropriate anatomical location (e.g., within the right or left atrium), the implant 80, the anchor launchers 74, and the support arms 72 (or at least a portion of each of the support arms 72) are extended distally beyond the distal end of the access sheath 60. The support arms 72 can then spread apart from each other, optionally with the help of the inflatable balloon 105, until the support arms 72 support the implant 80 so that the shape of the implant 80 approximates the shape of the annulus, as depicted in FIG. 1. Note that while FIG. 1 depicts four anchor launchers 74 and four support arms 72, it is preferable to use eight or more support arms 72, each of which supports a respective anchor launcher 74, each of which launches a respective anchor that is affixed to the implant 80.

The position of the implant 80 with respect to the annulus can then be adjusted by advancing or retracting the catheter 50 with respect to the access sheath 60 and/or rotating the catheter 50 radially with respect to the access sheath 60. If an actuatable bending section 52 is incorporated into the catheter 50, then actuating that bending section 52 will also adjust the position of the implant 80 with respect to the annulus prior to implantation. The actuatable bending section 52 may be constructed using any of a variety of conventional approaches that will be apparent to persons skilled in the art, including but not limited to the pull-wire based approaches described in U.S. Pat. Nos. 7,955,298 and 8,391,957, each of which is incorporated herein by reference. The pull wires run longitudinally through the catheter 50 from the bending section 52 back to the proximal end of the catheter, so that they can be manipulated by an operator using an appropriate set of controls.

Incorporating a plurality of degrees of freedom into the actuatable bending section 52 provides a significant amount of maneuverability, which is useful for aligning the implant 80 with the annulus prior to implantation. But depending on the anatomical context and/or the anatomy of a given individual subject, it can sometimes be difficult to align the implant 80 to the annulus with sufficient precision prior to implantation. One factor that contributes to this difficulty is the significant distance between the bending section 52 of the catheter 50 and the implant 80. For example, in the context depicted in FIG. 1 (i.e., delivering the implant to the mitral valve annulus), the bending section 52 of the catheter 50 could be more than 5 cm away from the implant 80, which can make it difficult to align the implant 80 with the annulus.

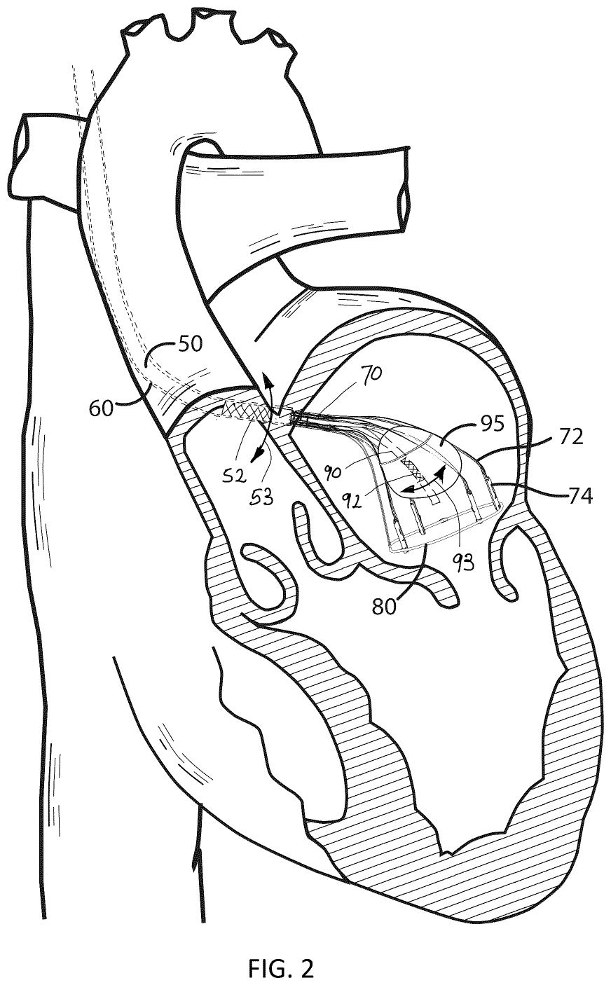

FIG. 2 depicts an embodiment that improves the maneuverability of the implant 80 with respect to the FIG. 1 approach, to make it easier to align the implant 80 with the annulus. The improvement in maneuverability is provided by incorporating a secondary actuatable bending section 92 into the shaft 90 that supports the inflatable balloon 95.

The FIG. 2 embodiment is an apparatus for affixing an implant 80 (e.g., a ring or a constricting cord) to a cardiac annulus or adjacent tissue. The apparatus includes a steerable catheter 50 having a distal portion, a distal end, and a first actuatable bending section 52 disposed in the distal portion of the catheter. Any of the approaches described above in connection with FIG. 1 for constructing the steerable catheter 50 and its bending section 52 may be used in this FIG. 2 embodiment.

A support structure 70 is affixed to the distal end of the catheter 50, and at least four support arms 72 are affixed to the support structure 70. Each of these support arms 72 extends distally beyond the support structure 70, and each of the support arms is at least 2 cm long. The construction of the support arms 72 and the support structure 70, and the affixation of the support structure 70 to the distal end of the catheter 50 may be implemented as described in U.S. Pat. No. 10,786,356. Alternatively, a variety of other approaches that will be apparent to persons skilled in the relevant arts may be used. A set of anchors are pre-affixed to the implant 80, and each of these anchors is driven into the annulus or adjacent tissues by a respective anchor launcher 74 that is supported by a respective one of the support arms 72. Note that while FIG. 2 depicts four anchor launchers 74 and four support arms 72, it is preferable to use eight or more support arms 72, each of which supports a respective anchor launcher 74, each of which launches a respective anchor that is affixed to the implant 80.

A shaft 90 extends distally from the support structure 70, and the shaft has a second actuatable bending section 92 positioned between the at least four support arms 72. The shaft 90 and the second bending section 92 may be constructed using approaches similar to conventional catheters and conventional bending sections. For example, the bending section 92 may be constructed using a plurality of vertebrae arranged to bend with a single degree of freedom in response to actuation of one or more pull wires.

Any suitable rigid interconnection may be used to affix the shaft 90 to the support structure 70. For example, the shaft 90 and the support structure 70 may be connected by providing a plurality of thin metal rods that extend distally from the support structure 70, and fitting those metal rods into corresponding lumens built into the proximal end of the shaft 90. In alternative embodiments, the interconnection between the shaft 90 and the support structure 70 may not be completely rigid. But a sufficient degree of stiffness at the interconnection between the shaft 90 and the support structure 70 is preferable in order to improve the maneuverability of the implant 80.

An inflatable balloon 95 surrounds at least a portion of the shaft 90, and at least a portion of the balloon 95 is positioned between the at least four support arms 72. The balloon 95 is inflatable using a conventional inflation lumen, and the balloon is configured so that inflation of the balloon 95 causes the balloon to press outward against all of the support arms 72 (or at least some of the support arms 72). Note that the outward pressure of the balloon 95 against the support arms 72 may be achieved when the outer surface of the balloon 95 presses directly against the support arms 72. Alternatively, the outward pressure of the balloon 95 against one or more of the support arms 72 may be achieved when the outer surface of the balloon 95 presses against another component (e.g., one of the anchor launchers 74) that is affixed to one of the support arms 72.

As noted above, the shaft 90 has a second actuatable bending section 92 positioned between the at least four support arms 72, the balloon 95 surrounds at least a portion of the shaft 90, and at least a portion of the balloon 95 is also positioned between the at least four support arms 72. When the balloon 95 is inflated, it presses outward against the support arms 72, and the support arms 72 support the implant 80. As a result of this sequential arrangement of components, actuating that bending section 92 will bend the shaft 90, which will move the inflated balloon 95, which will move at least some of the support arms 72, which will move the implant 80.

Because the second actuatable bending section 92 is located closer to the implant 80 (as compared to the first actuatable bending section 52), the second actuatable bending section 92 can be used to provide a fine adjustment of the position of the implant 80, while the first actuatable bending section 52 provides a coarser adjustment of the position of the implant 80. Using two actuatable bending section 52, 92, especially when one of them is located relatively close to the implant 80, provides a significant improvement in adjustability with respect to the FIG. 1 approach.

In these embodiments, the position of the implant 80 with respect to the annulus can be adjusted by advancing or retracting the catheter 50 with respect to the access sheath 60 and/or rotating the catheter 50 radially with respect to the access sheath 60. The actuatable bending section 52 (which is actuated, e.g., using pull wires) provides the least one and more preferably at least two degrees of freedom for coarse adjustments of the position of the implant 80. And the actuatable bending section 92 (which is also actuated, e.g., using pull wires) provides an additional degree of freedom for fine adjustments of the position of the implant 80.

In some embodiments, the first actuatable bending section 52 has at least two degrees of freedom and the second actuatable bending section 92 has at least one degree of freedom. In the context of delivering an implant to the tricuspid valve annulus or the mitral valve annulus, it can be beneficial to align those degrees of freedom so that the first actuatable bending section 52 and the second actuatable bending section 92 are both bendable in the same plane (e.g., the plane of the page in FIG. 2).

The alignment between the degrees of freedom within the first and second actuatable bending section 52, 92 may be implemented by providing the first actuatable bending section 52 with at least one actuator (e.g., two pull wires) configured to bend the first actuatable bending section 52 within a first plane (e.g., within the plane of the page in FIG. 2, as indicated by the double-headed arrow 53) and at least one actuator (e.g., two additional pull wires) configured to bend the first actuatable bending section 52 within a second plane that is offset by 60-120° (and more preferably 85-95°) from the first plane (e.g., in or out of the page in FIG. 2). In these embodiments, the second actuatable bending section 92 has at least one actuator (e.g., two additional pull wires) configured to bend the second actuatable bending section 92 within the first plane (e.g., within the plane of the page, as indicated by the double-headed arrow 93).

Note that FIG. 2 depicts the apparatus at a moment in time shortly before implantation of the implant 80 (i.e., before the anchor launchers 74 drive the anchors into the annulus). But prior to arriving at this state, the support arms 72, the anchor launchers 74, the flexible implant 80, and the shaft 90 are all squeezed together within an access sheath 60, so that they can be delivered via a minimally invasive approach. The access sheath 60 is dimensioned to slidably surround the catheter 50, the at least four support arms 72, the shaft 90, and the balloon 95. After the apparatus is routed to the appropriate anatomical location (e.g., within the right or left atrium), the implant 80, the anchor launchers 74, the shaft 90, the balloon 95, and the support arms 72 (or at least a portion of each of the support arms 72) are extended distally beyond the distal end of the access sheath 60. The support arms 72 may be configured to spring apart from each other upon sliding distally beyond a distal end of the access sheath 60, optionally with the assistance of the inflatable balloon 95, until the support arms 72 support the implant 80 so that the shape of the implant 80 approximates the shape of the annulus, as depicted in FIG. 2.

Optionally, a supplemental sheath (not shown) that initially surrounds the at least four support arms 72, the shaft 90, and the balloon 95 may be included in the FIG. 2 embodiment. This supplemental sheath is initially disposed between the components 72, 90, and 95 and the access sheath 60, and is concentric with the access sheath 60. When the supplemental sheath is included, it must be withdrawn so that the at least four support arms 72, the shaft 90, and the balloon 95 also extend distally beyond the distal end of the supplemental sheath before the support arms 72 will spring apart from each other.

In some embodiments, the implant 80 is either a ring or a constricting cord. In these embodiments, at least four anchors (not shown) are configured to affix the ring or cord to the cardiac annulus or adjacent tissue. At least four anchor launchers 74 are provided, each of which is configured to drive a respective one of the anchors into the cardiac annulus or adjacent tissue. Each of the anchor launchers 74 is supported by a respective one of the at least four support arms 72. Preferably, there are at least eight anchors and eight anchor launchers 74, each of which is supported by a respective one of at least eight support arms 72.

Although the FIG. 2 embodiment described above provides improved maneuverability of the implant 80 with respect to the FIG. 1 approach, the FIG. 2 embodiment does have one disadvantage with respect to FIG. 1. More specifically, because the shaft 90 includes an actuatable bending section 92 that must be able to exert enough force to deflect the support arms 72, most practical implementations of the shaft 90 will have a non-negligible thickness (e.g., 3 mm in diameter). And because the shaft 90, the balloon 95, the at least four support arms 72, and/or the implant 80 can all overlap at certain longitudinal positions along the device when the device is collapsed within the access sheath 60, the thickness of the shaft 90 can make a substantial contribution to the overall diameter of the device at those longitudinal positions. In these embodiments, the substantial contribution of the shaft 90 may prevent the device from fitting within an access sheath with the preferred outer diameter for the anatomic location (e.g., <8 mm for access to the right atrium via the superior vena cava).

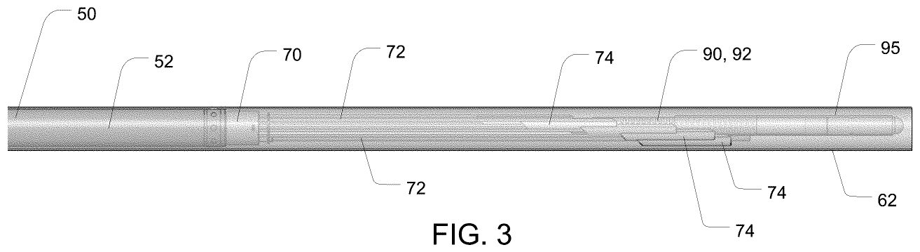

The embodiments described below in connection with FIGS. 3-10 avoid this disadvantage by positioning the secondary actuatable bending section in a subassembly that is positioned distally beyond the support structure that holds the support arms 72 and is also slidably disposed with respect to the support structure. When the subassembly is in its initial position, the shaft 90 and the balloon 95 are positioned distally beyond at least some of the support arms 72, the anchor launchers 74, and/or the implant 80, so there will be fewer components at certain longitudinal positions. This configuration can advantageously reduce the overall diameter of the device, so that it will be able to fit within an access sheath of the desired size and reach the target location in the subject's anatomy (e.g., the right atrium or the left atrium). After the device reaches the target location, the subassembly slides in the proximal direction (e.g., until the balloon is disposed between the at least four support arms). The subassembly then forms a keyed connection with the rest of the device, after which the overall device provides the same functionality as the FIG. 2 embodiment.

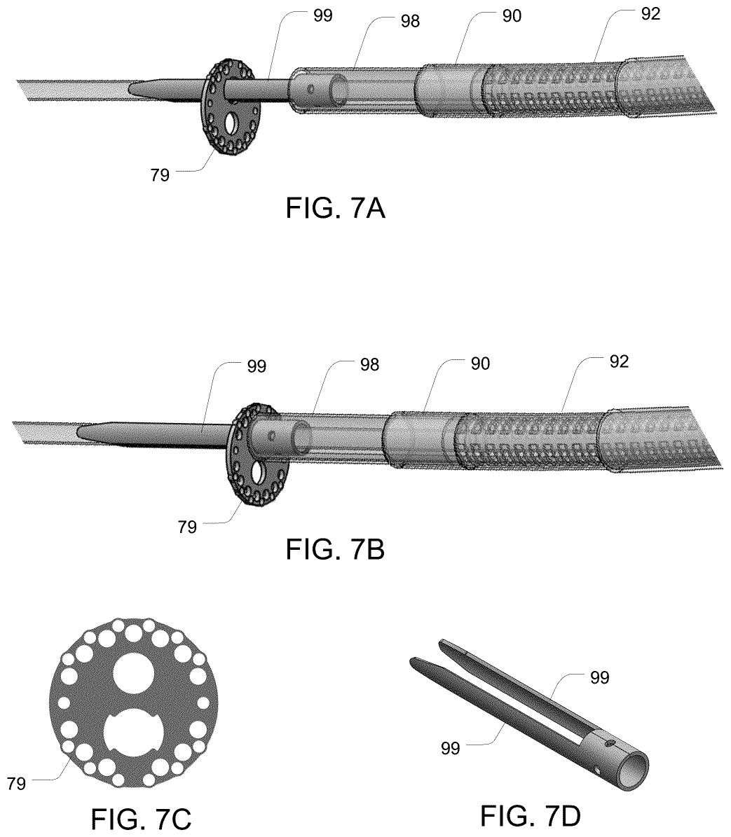

FIG. 3 depicts this embodiment at an initial point in time during which all of the components are squeezed together within a sheath 62 so that the implant (e.g., a ring or a cord) can be delivered through the subject's vasculature via a minimally-invasive approach e.g., to a cardiac annulus or adjacent tissue. This embodiment includes a steerable catheter 50 that has a distal portion, a distal end, and a first actuatable bending section 52 that is disposed in the distal portion of the catheter. A support structure 70 is affixed to the distal end of the catheter 50, and this support structure 70 includes at least one first mating feature (described below in connection with FIG. 7A-7D). At least four support arms 72 are affixed to the support structure 70, and these support arms 72 extend distally beyond the support structure. Each of the support arms is at least 2 cm long. The construction of the support arms 72 and the support structure 70 may be as described above in connection with FIG. 2

A set of at least four anchors are pre-affixed to the implant (e.g., the ring or cord), and each of these anchors is driven into the annulus or adjacent tissues by a respective anchor launcher 74 that is supported by a respective one of the support arms 72. Each of the at least four anchors is configured to affix the ring or cord to the cardiac annulus or adjacent tissue, and each of at least four anchor launchers is configured to drive a respective one of the anchors into the cardiac annulus or adjacent tissue. Some preferred embodiments use eight or more support arms 72, each of which supports a respective anchor launcher 74, each of which launches a respective anchor that is affixed to the implant 80.

This embodiment also includes a subassembly having a proximal segment and a proximal end. The subassembly includes a shaft 90 that extends distally from the proximal segment, and the shaft 90 has a second actuatable bending section 92. The shaft 90 and the second bending section 92 may be constructed using approaches similar to conventional catheters and conventional bending sections. For example, the bending section 92 may be constructed using a plurality of vertebrae arranged to bend with a single degree of freedom in response to actuation of one or more pull wires. In some embodiments, the shaft 90 is at least 3 cm long. Note that the shaft 90 and the proximal segment could be implemented as sub-regions of a single unitary component. Alternatively, the shaft 90 and the proximal segment could be implemented using two discrete components that are affixed together.

The subassembly also includes an inflatable balloon 95 that surrounds at least a portion of the shaft 90. The proximal segment of the subassembly has at least one second mating feature (described below in connection with FIG. 7A-7D) that is shaped and dimensioned to form a keyed connection with the at least one first mating feature. The subassembly is positioned distally beyond the support structure 70 and is slidably disposed with respect to the support structure. The support structure and the subassembly are disposed at respective initial positions at which the proximal end of the subassembly is spaced apart from the support structure by at least 1 cm. (This initial spacing is most clearly visible in FIG. 7A.) In some embodiments, the support structure and the subassembly are disposed at respective initial positions at which the proximal end of the subassembly is spaced apart from the support structure by at least 3 cm. Note that when measuring any of the spacings specified herein between the proximal end of the subassembly and the support structure, any male mating features that may extend from the subassembly or the support structure 70 should be ignored.

Initially, the support arms 72, the anchor launchers 74, and the shaft 90 are all squeezed together within a sheath 62 so that the implant (e.g., a ring or a cord) can be delivered through the subject's vasculature. This sheath 62 is dimensioned to slidably surround the catheter 50, the at least four support arms 72, and the subassembly. And all of these components (including the sheath 62) may be introduced into the subject's body by sliding them in a distal direction through an access sheath (not shown in FIG. 3) that is in turn dimensioned to slidably surround the sheath 62 (as well as the catheter 50, the at least four support arms 72, and the subassembly within that sheath 62).

The at least four support arms 72 are configured to spring apart from each other e.g., by making the support arms 72 from a shape memory material such as nitinol. But as long as the support arms 72 are confined within at least one of the sheath 62 and the access sheath that surrounds the sheath 62, the support arms remain squeezed together.

After all the components depicted in FIG. 3 have been advanced to the desired position in the subject's body, the sheath 62 is withdrawn so that it no longer covers the implant or the anchor launchers 74, and the operator slides the implant, the subassembly, the anchor launchers 74, and at least a portion of the support arms 72 distally beyond the distal end of the access sheath.

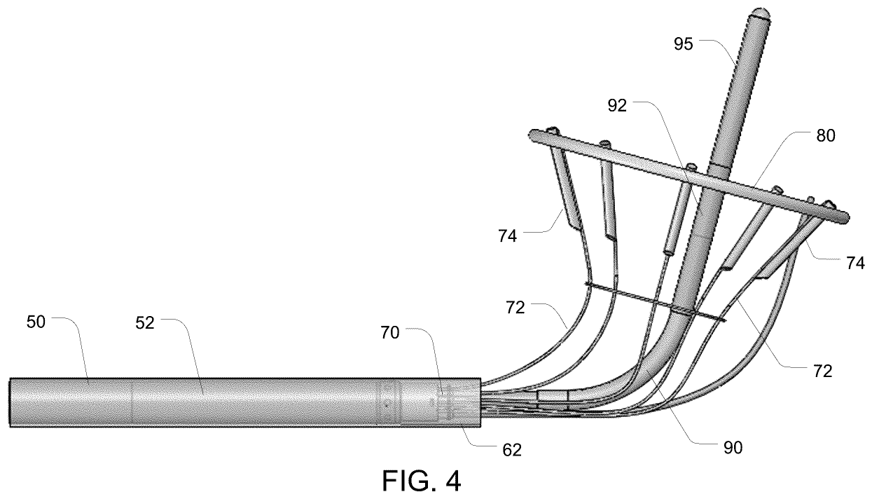

FIG. 4 depicts the state of the device after the sheath 62 is withdrawn and the operator slides the implant 80, the subassembly, the anchor launchers 74, and most of the support arms 72 distally beyond the distal end of the sheath 62 and the access sheath. More specifically, the support arms 72 will spring apart as soon as they have slid distally beyond both the sheath 62 and the access sheath. And because anchors are affixed to the implant 80, and those anchors are housed in the anchor launchers 74 which are in turn supported by the support arms 72, when those components exit the distal end of the access sheath, the spring action of the support arms 72 will expand the implant 80 to an expanded state (e.g., as depicted in FIG. 4).

As explained above, the subassembly (which includes the shaft 90 and the inflatable balloon 95) is initially positioned distally beyond the support structure 70 at respective initial positions at which the proximal end of the subassembly is spaced apart from the support structure by at least 1 cm. The subassembly is configured so that it can slide in a proximal direction with respect to the catheter 50 to a second position after the support arms have sprung apart from each other.

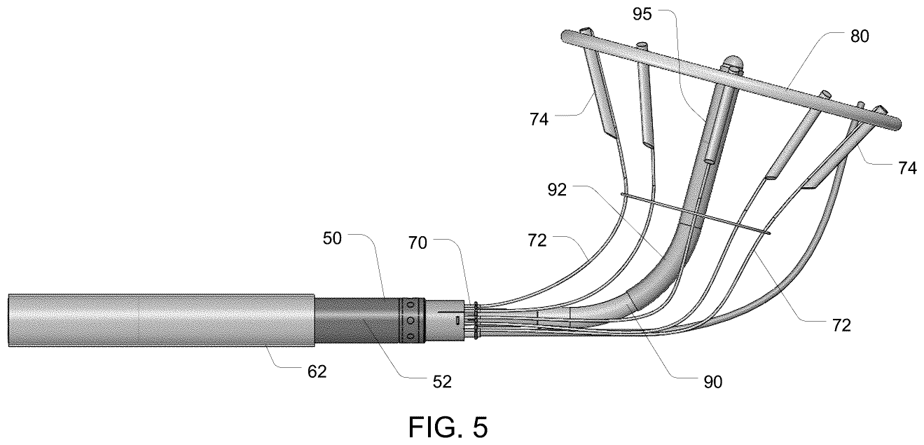

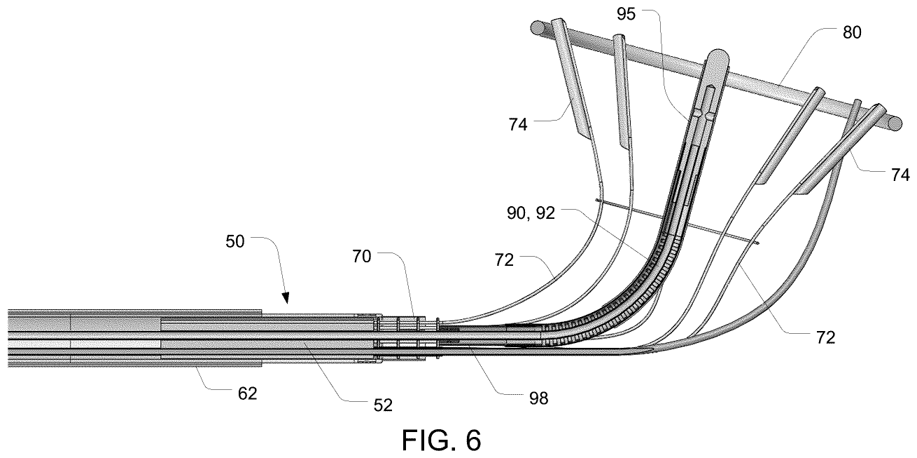

FIG. 5 depicts the state of the device after the subassembly has slid in the proximal direction with respect to the catheter 50, and FIG. 6 is a corresponding cross-sectional view. The positions of all of the components visible in FIG. 5 are the same as in FIG. 4 (discussed above) except for the subassembly (which includes the shaft 90 and the inflatable balloon 95). More specifically, the subassembly has moved by sliding in the proximal direction with respect to FIG. 4 until the subassembly reaches a second position (i.e., the position depicted in FIG. 5). Note that when the subassembly is at the second position, the distal end of the subassembly does not extend as far beyond the implant 80 (as compared to the pre-sliding position depicted in FIG. 4). When the subassembly has slid to the second position, at least a portion of the balloon 95 is disposed between the at least four support arms 72.

As noted above, the support structure 70 includes at least one first mating feature, and the proximal segment of the subassembly has at least one second mating feature that is shaped and dimensioned to form a keyed connection with the at least one first mating feature. When the subassembly slides to the second position, the at least one second mating feature makes the keyed connection with the at least one first mating feature.

FIGS. 7A-7D depict one example of a set of first and second mating features that may be used to make a keyed connection between the subassembly and the support structure 70. More specifically, FIG. 7A is a detail of the interface between the subassembly and the distalmost component 79 of the support structure 70 when the device is in the initial state depicted in FIG. 4; and FIG. 7B is a detail of that interface in the state depicted in FIG. 5 (i.e., after the subassembly has moved to the second position). FIG. 7C depicts a detail of two openings (i.e., female mating features) that are designed into the distalmost component 79 of the support structure 70. These openings serve as the first mating features. And FIG. 7D depicts a detail of two male prongs (i.e., the second mating features) that are shaped and dimensioned to slidably fit into the plurality of openings and form a keyed connection with the first mating features.

In both FIG. 4 and FIG. 7A, the support structure and the subassembly are disposed at respective initial positions at which the proximal end of the subassembly 98 is spaced apart from the distalmost component 79 of the support structure 70 by at least 1 cm, and the second mating features have not yet formed a keyed connection with first mating features. When the subassembly slides in the proximal direction and reaches the second position, the proximal end of the subassembly 98 will have moved closer to the support structure 70 (e.g., until it butts up against the distalmost component 79 of the support structure 70, as depicted in both FIG. 5 and FIG. 7B). The interaction between the female first mating features of the distalmost component 79 of the support structure 70 and the male second mating features 99 forms a keyed connection between the subassembly and the support structure 70.

A variety of alternative approaches for making the keyed connection between the subassembly and the support structure 70 may be used in place of the prong/opening approach depicted in FIGS. 7A-7D. For example, a pair of lumens may be built into the support structure 70, and a pair of flexible metal rods that are dimensioned to slide into those lumens may extend back in a proximal direction from the subassembly. In alternative embodiments, the sex of the first and second mating features may be reversed, in which case female mating features would be built into the subassembly, and male mating features would be affixed to the support structure 70 (and extend distally beyond the support structure 70).

Returning to FIG. 5, when the subassembly has slid to the second position, at least a portion of the balloon 95 is disposed between the at least four support arms 72. The balloon 95 is inflatable e.g., using a conventional inflation lumen. And the balloon 95 is configured so that when the subassembly is at the second position, inflation of the balloon 95 causes the balloon to press outward against all of the support arms 72 (or at least some of the support arms 72).

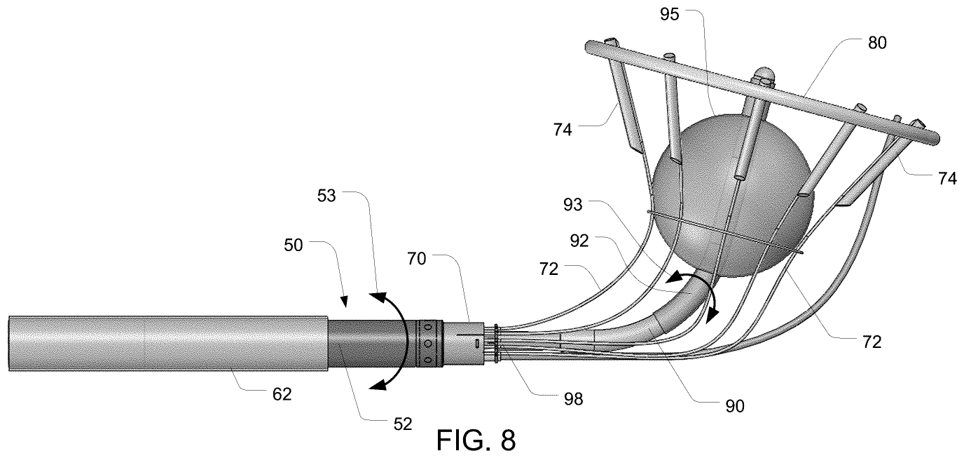

FIG. 8 depicts the state of the device after the balloon 95 has been inflated, and the balloon is pressing outward against all of the support arms 72. As in the FIG. 2 embodiment, the outward pressure of the balloon 95 against the support arms 72 may be achieved when the outer surface of the balloon 95 presses directly against the support arms 72. Alternatively, the outward pressure of the balloon 95 against one or more of the support arms 72 may be achieved when the outer surface of the balloon 95 presses against another component (e.g., one of the anchor launchers 74) that is affixed to one of the support arms 72.

Because the balloon is pressing outward against at least some of the support arms 72, and the support arms 72 support the implant 80, and because the balloon 95 surrounds at least a portion of the shaft 90, actuating that bending section 92 will bend the shaft 90, which will move the inflated balloon 95, which will move at least some of the support arms 72, which will move the implant 80.

Because the second actuatable bending section 92 is located closer to the implant 80 (as compared to the first actuatable bending section 52), the second actuatable bending section 92 can be used to provide a fine adjustment of the position of the implant 80, while the first actuatable bending section 52 provides a coarser adjustment of the position of the implant 80. Using two actuatable bending section 52, 92, especially when one of them is located relatively close to the implant 80, provides a significant improvement in adjustability with respect to the FIG. 1 approach.

The position of the implant 80 with respect to the annulus can be adjusted by advancing or retracting the catheter 50 with respect to the access sheath and/or rotating the catheter 50 radially with respect to the access sheath. The actuatable bending section 52 (which is actuated, e.g., using pull wires) provides at least one and more preferably at least two degrees of freedom for coarse adjustments of the position of the implant 80. And the actuatable bending section 92 (which is also actuated, e.g., using pull wires) provides an additional degree of freedom for fine adjustments of the position of the implant 80.

In some embodiments, the first actuatable bending section 52 has at least two degrees of freedom and the second actuatable bending section 92 has at least one degree of freedom. In the context of delivering an implant to the tricuspid valve annulus or the mitral valve annulus, it can be beneficial to align those degrees of freedom so that the first actuatable bending section 52 and the second actuatable bending section 92 are both bendable in the same plane, e.g., as described above in connection with the FIG. 2 embodiment.

The alignment between the degrees of freedom within the first and second actuatable bending section 52, 92 may be implemented by providing the first actuatable bending section 52 with at least one actuator (e.g., two pull wires) configured to bend the first actuatable bending section 52 within a first plane (e.g., within the plane of the page in FIG. 8, as indicated by the double-headed arrow 53) and at least one actuator (e.g., two additional pull wires) configured to bend the first actuatable bending section 52 within a second plane that is offset by 60-120° (and more preferably 85-95°) from the first plane (e.g., in or out of the page in FIG. 2). In these embodiments, the second actuatable bending section 92 has at least one actuator (e.g., two additional pull wires) configured to bend the second actuatable bending section 92 within the first plane (e.g., within the plane of the page, as indicated by the double-headed arrow 93).

The FIGS. 3-10 embodiment provides improved maneuverability of the implant 80 with respect to the FIG. 1 approach, and the same maneuverability as the FIG. 2 embodiment. But the FIGS. 3-10 embodiment provides a significant advantage over the FIG. 2 embodiment. More specifically, because the subassembly that includes the shaft 90 and the balloon 95 is longitudinally slidable with respect to the catheter 50 and the support structure the entire device can fit into a smaller access sheath than the FIG. 2 embodiment. This is because the shaft 90 and balloon 95 can initially be positioned in a non-working position distally beyond at least some of the support arms 72 and the anchor launchers 74 while the device is advanced within the access sheath through the subject's vasculature, which means that fewer components line up at certain longitudinal positions. This advantageously reduces the overall diameter of the device at those longitudinal positions, which helps the device fit within an access sheath with the preferred outer diameter (e.g., <8 mm) for the anatomic location.

The subassembly is only retracted to its working position (i.e., the mated position where at least a portion of the balloon is disposed between the at least four support arms) after the device has exited the distal end of the access sheath and entered the atrium. But due to the relative spaciousness of both the right and left atria, the overall diameter of the device when the subassembly is retracted to its working position is no longer a limiting factor at this point of the procedure.

After the position of the implant 80 has been adjusted to conform with the annulus (e.g., by advancing or retracting the catheter 50, rotating the catheter 50 radially, or actuating the first and second actuatable bending sections 52, 92 as described above), the anchor launchers 74 drive the anchors into the annulus, which affixes the implant 80 to the annulus.

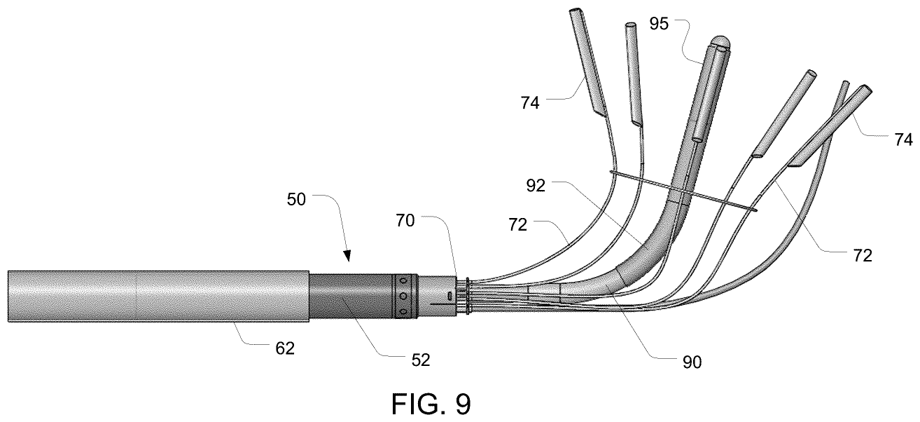

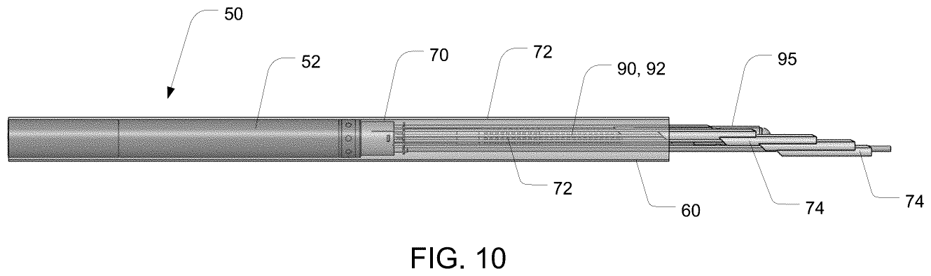

FIG. 9 depicts the state of the device after the implant has been affixed to the annulus. At this point, the implant is no longer connected to the anchor launchers 74. The catheter 50, the support structure 70, the support arms 72, the anchor launchers 74, the shaft 90, and the balloon 95 must now all be withdrawn from the subject's body. This can be accomplished by pulling all those components in a proximal direction through the access sheath 60 (i.e., the same access sheath through which they were originally introduced), as depicted in FIG. 10. More specifically, FIG. 10 depicts the catheter 50, the support structure 70, the support arms 72, the anchor launchers 74, the shaft 90, and the balloon 95 being pulled in a proximal direction through the access sheath 60 at a moment in time where portions of the support arms 72, some of the anchor launchers 74, and most of the shaft 90 have already entered the access sheath 60. All of those components can then be withdrawn in the proximal direction through the access sheath.

Note that because the implant is no longer attached to the anchor launchers 74 at this point in the procedure, the support arms 72 and the anchor launchers 74 can compress more closely (as compared to the situation in FIG. 3 when the implant was still attached to the anchor launchers 74). In view of this increased compression, all the components 50, 70, 72, 74, 90, 95 can be withdrawn through the access sheath 60 despite the fact that the subassembly remains mated with the support structure 70. But alternatively (e.g., at the discretion of the operator), the subassembly may be advanced in the distal direction with respect to the support structure 70 (e.g., back to its initial position) before the entire device is withdrawn through the access sheath 60.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for affixing a ring or cord to a cardiac annulus or adjacent tissue, the apparatus comprising:
a steerable catheter having a distal portion, a distal end, and a first actuatable bending section disposed in the distal portion of the catheter;
a support structure affixed to the distal end of the catheter, wherein the support structure includes at least one first mating feature;
at least four support arms, each of which is affixed to the support structure and extends distally beyond the support structure, wherein each of the support arms is at least 2 cm long; and
a subassembly having a proximal segment and a proximal end, wherein the subassembly includes
a shaft that extends distally from the proximal segment, wherein the shaft has a second actuatable bending section, and
an inflatable balloon that surrounds at least a portion of the shaft,
wherein the proximal segment of the subassembly has at least one second mating feature that is shaped and dimensioned to form a keyed connection with the at least one first mating feature,
wherein the subassembly is positioned distally beyond the support structure and is slidably disposed with respect to the support structure, and
wherein the support structure and the subassembly are disposed at respective initial positions at which the proximal end of the subassembly is spaced apart from the support structure by at least 1 cm.

2. The apparatus of claim 1
wherein the at least four support arms are configured to spring apart from each other upon sliding distally beyond a distal end of a sheath that is dimensioned to slidably surround the catheter, the at least four support arms, and the subassembly,
wherein the subassembly is configured for sliding in a proximal direction with respect to the catheter to a second position after the support arms have sprung apart from each other, such that when the subassembly has slid to the second position, (a) the at least one second mating feature makes the keyed connection with the at least one first mating feature and (b) at least a portion of the balloon is disposed between the at least four support arms, and
wherein the balloon is configured so that when the subassembly is at the second position, inflation of the balloon causes the balloon to press outward against at least some of the support arms.

3. The apparatus of claim 2 further comprising the sheath.

4. The apparatus of claim 1, wherein the at least one first mating feature comprises at least one opening, and wherein the at least one second mating feature comprises at least one member dimensioned to slidably fit into the at least one opening.

5. The apparatus of claim 1, wherein the at least one first mating feature comprises a plurality of openings, and wherein the at least one second mating feature comprises a plurality of prongs dimensioned to slidably fit into the plurality of openings.

6. The apparatus of claim 1, wherein the at least one first mating feature comprises at least two lumens, and wherein the at least one second mating feature comprises at least two flexible metal rods dimensioned to slide within the at least two lumens.

7. The apparatus of claim 1, wherein the first actuatable bending section has at least two degrees of freedom and wherein the second actuatable bending section has at least one degree of freedom.

8. The apparatus of claim 1, wherein the first actuatable bending section has at least one actuator configured to bend the first actuatable bending section within a first plane and at least one actuator configured to bend the first actuatable bending section within a second plane that is offset by 60-120° from the first plane, and
wherein the second actuatable bending section has at least one actuator configured to bend the second actuatable bending section within the first plane.

9. The apparatus of claim 1, wherein the first actuatable bending section has at least one pull wire configured to bend the first actuatable bending section within a first plane and at least one pull wire configured to bend the first actuatable bending section within a second plane that is offset by 60-120° from the first plane, and
wherein the second actuatable bending section has at least one pull wire configured to bend the second actuatable bending section within the first plane.

10. The apparatus of claim 1, wherein the support structure and the subassembly are disposed at respective initial positions at which the proximal end of the subassembly is spaced apart from the support structure by at least 3 cm.

11. The apparatus of claim 1, wherein the shaft is at least 3 cm long.

12. The apparatus of claim 1, wherein the catheter has an outer diameter less than 8 mm.

13. The apparatus of claim 1, further comprising:
the ring or cord;
at least four anchors configured to affix the ring or cord to the cardiac annulus or adjacent tissue; and
at least four anchor launchers, each of which is configured to drive a respective one of the anchors into the cardiac annulus or adjacent tissue,
wherein each of the at least four anchor launchers is supported by a respective one of the at least four support arms.

14. An apparatus for affixing a ring or cord to a cardiac annulus or adjacent tissue, the apparatus comprising:
a steerable catheter having a distal portion, a distal end, and a first actuatable bending section disposed in the distal portion of the catheter;
a support structure affixed to the distal end of the catheter;
at least four support arms, each of which is affixed to the support structure and extends distally beyond the support structure, wherein each of the support arms is at least 2 cm long;
a shaft that extends distally from the support structure, wherein the shaft has a second actuatable bending section positioned between the at least four support arms; and
an inflatable balloon that surrounds at least a portion of the shaft, wherein at least a portion of the balloon is positioned between the at least four support arms.

15. The apparatus of claim 14
wherein the at least four support arms are configured to spring apart from each other upon sliding distally beyond a distal end of a sheath that is dimensioned to slidably surround the catheter, the at least four support arms, the shaft, and the balloon, and
wherein the balloon is configured so that inflation of the balloon causes the balloon to press outward against at least some of the support arms.

16. The apparatus of claim 15 further comprising the sheath.

17. The apparatus of claim 14, wherein the first actuatable bending section has at least two degrees of freedom and wherein the second actuatable bending section has at least one degree of freedom.

18. The apparatus of claim 14, wherein the first actuatable bending section has at least one actuator configured to bend the first actuatable bending section within a first plane and at least one actuator configured to bend the first actuatable bending section within a second plane that is offset by 60-120° from the first plane, and
wherein the second actuatable bending section has at least one actuator configured to bend the second actuatable bending section within the first plane.

19. The apparatus of claim 14, wherein the first actuatable bending section has at least one pull wire configured to bend the first actuatable bending section within a first plane and at least one pull wire configured to bend the first actuatable bending section within a second plane that is offset by 60-120° from the first plane, and
wherein the second actuatable bending section has at least one pull wire configured to bend the second actuatable bending section within the first plane.

20. The apparatus of claim 14, further comprising:
the ring or cord;
at least four anchors configured to affix the ring or cord to the cardiac annulus or adjacent tissue; and
at least four anchor launchers, each of which is configured to drive a respective one of the anchors into the cardiac annulus or adjacent tissue,
wherein each of the at least four anchor launchers is supported by a respective one of the at least four support arms.
